(19) United States
(12) Patent Application Publication
(10) Pub. No.: US 2006/0235245 A1
(43) Pub. Date: Oct. 19, 2006

(54) Process for producing oxygen-bearing compound

(75) Inventors: Ichiro Yamanaka, Tokyo (JP); Kiyoshi Otsuka, Saitama (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignees: Idemitsu Kosan Co., Ltd., Chiyoda-ku (JP); Tokyo Institute of Technology, Meguro-ku (JP)

(21) Appl. No.: 11/367,354

(22) Filed: Mar. 6, 2006

(30) Foreign Application Priority Data
Mar. 14, 2005 (JP) 2005-070611

(51) Int. Cl. C07C 35/22; C07C 45/33

(52) U.S. Cl. 568/373; 568/400; 568/910; 568/818

(57) ABSTRACT

Provided is a process for industrially advantageously producing an oxygen-bearing compound (e.g., an alcohol, a diol, a polyol, or a ketone) through oxidation of an alkane or an alcohol, which process requires no treatment for separation/removal of a catalyst and causes no equipment corrosion. Specifically, there are provided a process for producing an oxygen-bearing compound, including oxidizing an alkane in the presence of a catalyst containing at least one element selected from among transition metal elements belonging to Groups 5 and 8 to 10 of the periodic table, wherein the oxygen-bearing compound is an alcohol, a diol, a polyol, or a ketone; and a process for producing an oxygen-bearing compound, including oxidizing an alcohol in the presence of a catalyst containing at least one element selected from among transition metal elements belonging to Groups 5 and 8 to 10 of the periodic table, wherein the oxygen-bearing compound is a diol, a polyol, or a ketone.

### TECHNICAL FIELD

The present invention relates to a process for producing an oxygen-bearing compound from a saturated hydrocarbon compound (alkane) or an alcohol derived therefrom, and more particularly to a process for conveniently producing an oxygen-bearing compound (e.g., an alcohol, a diol, a polyol, or a ketone) through partial oxidation of an alkane or an alcohol derived therefrom. Such an oxygen-bearing compound is useful as a solvent or as an intermediate for producing various chemical products.

### BACKGROUND ART

Oxidation processes employing molecular oxygen for achieving oxidation economical as air can be utilized, and thus they are considered industrially very important.

For example, adipic acid, which serves as a raw material of 6,6-nylon, and ε-caprolactam, which serves as a raw material of 6-nylon, are currently derived from cyclohexane, or cyclohexanol produced through oxidation of cyclohexane. Meanwhile, terephthalic acid, which serves as a raw material of polyethylene terephthalate, is produced through oxidation of p-xylene, and maleic anhydride is produced through oxidation of butane.

As mentioned above, a process for oxidation of cyclohexane, which is an alkane and an alicyclic hydrocarbon, is an industrially important technique, and hitherto, an oxidation technique employing cobalt naphthenate as a catalyst has been established. However, such a technique is not necessarily satisfactory in terms of, for example, conversion, yield, and reaction pressure.

In view of the foregoing, in recent years, there has been developed a cyclohexane oxidation process employing N-hydroxyphthalimide (NHPI) as a main catalyst, and a cobalt salt or a vanadium compound as a promoter (see, for example, Patent Documents 1 and 2). However, in this process, NHPI (i.e., catalyst) per se is decomposed through reaction, and NHPI and decomposition products thereof, which may be intermingled with a final product, must be separated from the reaction mixture. This technique is not yet satisfactory in terms of conversion, yield, and reaction pressure.

Therefore, demand has arisen for development of a cyclohexane oxidation technique which realizes lower reaction pressure and higher conversion and yield.

Adamantane, which is an alicyclic hydrocarbon, is known as a highly symmetric basket-shaped compound having the same structure as a diamond-structure unit cell. Adamantane has, for example, the following chemical features: (1) low molecular strain energy and high thermal stability; (2) high lipid solubility by virtue of high carbon density; and (3) sublimability but only slight odor. Since the 1980s, adamantane has become of interest in the field of pharmaceuticals as a raw material of therapeutic drugs for Parkinson's disease and influenza. In recent years, adamantane derivatives, which have heat resistance, transparency, and like characteristics, have become of interest in the fields of, for example, photoresists for semiconductor production; magnetic recording media; optical materials such as optical fibers, optical lenses, and optical disk substrate raw materials; functional materials such as heat-resistant plastic materials, coating materials, and adhesives; and cosmetics, and applications of the derivatives have been increasing. Also, adamantane derivatives have been increasing in demand in the field of pharmaceuticals as raw materials of, for example, anticancer drugs, brain function improving drugs, therapeutic drugs for nervous diseases, and antiviral drugs.

In recent years, adamantanol or adamantanone, which is a product obtained through oxidation of adamantane, has been rapidly increasing in demand as an intermediate for producing various functional materials.

Regarding production of such adamantane derivatives, for example, 2-adamantanone is produced through oxidation of adamantane by use of sulfuric acid, and adamantanol, 1,3-adamantanediol, and adamantanepolyol are produced through bromination and hydrolysis of adamantane.

Known techniques for selectively producing 1-adamantanol include a process for hydrolyzing bromoadamantane; an oxidation process employing hypochlorous acid as an oxidizing agent in the presence of a ruthenium chloride catalyst (see, for example, Patent Document 3); a process employing ozone as an oxidizing agent (see, for example, Patent Documents 4 and 5); and a process employing N-hydroxyphthalimide (NHPI) as a main catalyst, a cobalt salt or a vanadium compound as a promoter, and oxygen or air as an oxidizing agent (see, for example, Patent Documents 6 to 8). For example, Patent Document 6 discloses oxidation of adamantane in the presence of an NHPI/Co(acac)₂ (acetylacetonatocobalt(II)) catalyst or an NHPI/Co(acac)₃ (acetylacetonatocobalt(III)) catalyst, and Patent Document 8 discloses oxidation of adamantane in the presence of an NHPI/V₂O₅ catalyst or an NHPI/V(acac)₃ (acetylacetonatovanadium) catalyst. 1,3-Adamantanediol or an adamantanepolyol can be produced through a process for hydrolyzing dibromoadamantane (see, for example, Patent Document 9) or through sequential oxidation of 1-adamantanol, and Patent Document 9 describes such an adamantane derivative as one of main products. Known processes for producing 2-adamantanone include an oxidation process employing sulfuric acid (see, for example, Patent Document 10).

However, the bromination/hydrolysis process, which employs bromine as a reactant, incurs high raw material cost, as well as high construction cost for preventing equipment corrosion and leakage of a reaction product. The ruthenium chloride process requires recovery and recycling of the expensive catalyst, and raises a problem in that chlorinated adamantane is by-produced. The ozone process employs ozone (i.e., a highly toxic substance), and thus poses a problem in terms of safety. In the NHPI process, NHPI (i.e., catalyst) per se is decomposed through reaction, and NHPI and decomposition products thereof, which may be intermingled with a final product, must be separated from the reaction mixture. That is, the NHPI process requires treatment for separation/removal of the catalyst (i.e., a very cumbersome operation). The sulfuric acid process, which employs sulfuric acid as a catalyst and as a reaction solvent, requires a large amount of sulfuric acid, incurs high treatment cost for neutralizing the total amount of sulfuric acid, and imposes a high load on the environment.

In addition to the aforementioned processes, there have been proposed a process employing t-butyl hydroperoxide as an oxidizing agent and a metal complex as a catalyst (see, for example, Non-Patent Document 1) and a process employing hydrogen peroxide as an oxidizing agent and an iron compound or the like as a catalyst (see, for example, Non-Patent Document 2). However, such a proposed process requires an expensive oxidizing agent and encounters difficulty in controlling reaction, and therefore, industrialization of the process seems difficult. In recent years, it has also been reported that a vanadium(V)-on-montmorillonite catalyst is effective for oxidizing adamantane into adamantanol (see, for example, Non-Patent Document 3).

Non-Patent Document 3 reports that adamantane conversion is 93%, and 1-adamantanol selectivity, 2-adamantanone selectivity, and 1,3-adamantanediol selectivity are as high as 41%, 15%, and 43%, respectively. However, in this oxidation process, a long reaction time (96 hours) is required, and the turnover number is about 100. Thus, this process requires further improvement in terms of reaction rate. That is, this process employing a vanadium/montmorillonite catalyst poses problems in terms of industrialization. Therefore, a process for industrially producing 1-adamantanol, 1,3-adamantanediol, an adamantanepolyol, or 2-adamantanone through direct oxidation of adamantane by molecular oxygen has not been put into practice, and such a process has much room for improvement.

[Patent Document 1]

Japanese Patent Application Laid-Open (kokai) No. 2002-128714

[Patent Document 2]

Japanese Patent Application Laid-Open (kokai) No. 2002-161056

[Patent Document 3]

Japanese Patent Application Laid-Open (kokai) No. 2004-51497

[Patent Document 4]

Japanese Patent Application Laid-Open (kokai) No. 2004-189610

[Patent Document 5]

Japanese Patent Application Laid-Open (kokai) No. 2004-26778

[Patent Document 6]

Japanese Patent Application Laid-Open (kokai) No. 9-327626

[Patent Document 7]

Japanese Patent Application Laid-Open (kokai) No. 10-309469

[Patent Document 8]

Japanese Patent Application Laid-Open (kokai) No. 10-316601

[Patent Document 9]

Japanese Patent Application Laid-Open (kokai) No. 2000-327604

[Patent Document 10]

Japanese Patent Application Laid-Open (kokai) No. 11-189564

[Non-Patent Document 1]

J. Chem. Soc. Dalton Trans., 21, 1995, 3537-3542

[Non-Patent Document 2]

Chem. Pharm. Bull., 31, 4, 1983, 1166-1171

[Non-Patent Document 3]

Kaneda, et al., Annual Meeting of the Chemical Society of Japan, proceedings, 2003 and 2004

### DISCLOSURE OF THE INVENTION

Problems to be Solved by the Invention

An object of the present invention is to provide a process for industrially advantageously producing an oxygen-bearing compound (e.g., an alcohol, a diol, a polyol, or a ketone) through oxidation of an alkane or an alcohol derived therefrom in the presence of a new catalyst system, which process requires no intricate operation (i.e., treatment for separation/removal of the catalyst) and causes no equipment corrosion.

Means for Solving the Problems

The present inventors have conducted extensive studies for solving the aforementioned problems, and as a result have found that when a transition metal compound (e.g., a vanadium compound or a cobalt compound), which has conventionally been employed as a promoter of NHPI, is employed as a sole catalyst in a smaller amount, and direct partial oxidation is performed by means of oxygen or air in the presence of the catalyst, a target oxygen-bearing compound can be produced efficiently without requiring treatment for separation/removal of the catalyst. Specifically, the present inventors have found that even when a transition metal compound (e.g., a vanadium compound or a cobalt compound) is employed as a sole catalyst in a smaller amount, proper selection of reaction conditions attains high yield production of, for example, 1-adamantanol, 1,3-adamantanediol, adamantanepolyol, or 2-adamantanone from adamantane, or cyclohexanol or cyclohexanone from cyclohexane. The present invention has been accomplished on the basis of this finding.

Accordingly, the present invention provides a process for producing an oxygen-bearing compound, as described below.

1. A process for producing an oxygen-bearing compound, comprising oxidizing an alkane in the presence of a catalyst containing at least one element selected from among transition metal elements belonging to Groups 5 and 8 to 10 of the periodic table, wherein the oxygen-bearing compound is an alcohol, a diol, a polyol, or a ketone.

2. A process for producing an oxygen-bearing compound, comprising oxidizing an alcohol in the presence of a catalyst containing at least one element selected from among transition metal elements belonging to Groups 5 and 8 to 10 of the periodic table, wherein the oxygen-bearing compound is a diol, a polyol, or a ketone.

3. A process for producing an oxygen-bearing compound as described in 1 or 2 above, wherein the transition metal element contained in the catalyst is vanadium.

4. A process for producing an oxygen-bearing compound as described in 3 above, wherein vanadium contained in the catalyst has a valence of 3 to 5.

5. A process for producing an oxygen-bearing compound as described in 4 above, wherein the catalyst is a vanadium compound having a vanadium valence of 3 to 5, or a solid catalyst formed of a porous inorganic metallic carrier bearing the vanadium compound.

6. A process for producing an oxygen-bearing compound as described in 1 or 2 above, wherein the transition metal element contained in the catalyst is cobalt.

7. A process for producing an oxygen-bearing compound as described in 6 above, wherein the catalyst is a cobalt compound having a cobalt valence of 2 or 3, or a solid catalyst formed of a porous inorganic metallic carrier bearing the cobalt compound.

8. A process for producing an oxygen-bearing compound as described in 5 or 7 above, wherein the porous inorganic metallic carrier is formed of silica, alumina, silica-alumina, titania, silica-titania, zeolite, titanosilicate, mesoporous silica, or mesoporous titania.

9. A process for producing an oxygen-bearing compound as described in any of 1 through 8 above, wherein oxidation is carried out by use of a molecular-oxygen-containing gas serving as an oxidizing agent.

10. A process for producing an oxygen-bearing compound as described in any of 1 through 9 above, wherein oxidation is carried out in the co-presence of one or more species selected from among a carboxylic acid, a sulfonic acid, and a Lewis acid.

11. A process for producing an oxygen-bearing compound as described in any of 1 through 10 above, wherein the catalyst containing a transition metal element is employed as a sole catalyst in an amount of 0.00001 to 10 parts by mass on the basis of 100 parts by mass of raw material.

12. A process for producing an oxygen-bearing compound as described in any of 1 through 11 above, wherein the alkane is adamantane, the alcohol is 1-adamantanol or 2-adamantanol, the diol is 1,3-adamantanediol, the polyol is adamantanepolyol, and the ketone is 2-adamantanone.

Effects of the Invention

According to the present invention, when a transition-metal-containing catalyst is employed as a sole catalyst in a small amount, and oxidation of an alkane is performed in the presence of the catalyst under selected reaction conditions, an oxygen-bearing compound (e.g., an alcohol, a diol, a polyol, or a ketone) can be industrially produced with a high level of convenience and at low cost without requiring treatment for separation/removal of the catalyst. Meanwhile, when oxidation of an alcohol is performed in the presence of a transition-metal-containing catalyst in a manner similar to that described above, an oxygen-bearing compound (e.g., a diol, a polyol, or a ketone) can be industrially produced with a high level of convenience and at low cost without requiring treatment for separation/removal of the catalyst. Therefore, an expensive catalyst (e.g., ruthenium or NHPI) is not required, and catalyst cost can be reduced. Particularly, air or oxygen can be employed as an oxidizing agent, and therefore raw material cost can be reduced. In addition, since equipment corrosion which would otherwise occur when hydrogen peroxide, hypochlorous acid, or the like is employed as an oxidizing agent does not occur, no particular treatment is required for prevention of equipment corrosion, and thus equipment construction cost can be reduced.

Among oxygen-bearing compounds produced through the production process of the present invention, for example, cyclohexanol or cyclohexanone, which is produced from cyclohexane, is in very high demand as a raw material for nylon, which is a typical synthetic fiber. Meanwhile, 1-adamantanol, 1,3-adamantanediol (or an adamantanepolyol having three or more hydroxyl groups), or 2-adamantanone, which is produced from adamantane, is a compound which is highly useful as a raw material for electronic materials or as an intermediate for producing various chemical products including pharmaceuticals and agrichemicals.

In any of the processes disclosed in the aforementioned patent documents, reaction is performed in a homogenous system (i.e., a catalyst is uniformly dissolved in a reaction solution), and thus a large amount of energy is consumed for separation/recovery of the catalyst from the reaction mixture. In contrast, the production process of the present invention, which may employ a solid catalyst formed of a carrier impregnated with a vanadium compound or a cobalt compound, is advantageous in that regardless of whether fixed-bed reaction or batch-type reaction is performed, after completion of reaction, the catalyst is readily separated from the reaction mixture, and the catalyst is easily recycled. In the case of oxidation of adamantane in the presence of a vanadium/montmorillonite catalyst as disclosed in the Non-Patent Document 3, the valence of elemental vanadium serving as an active species is limited to 5, and industrially sufficient reaction rate (high turnover number) fails to be attained. In contrast, the present invention is industrially advantageous in that, for example, elemental vanadium having a wide range of valence (3 to 5) can be employed; a cobalt compound can be employed in addition to a vanadium compound; and high turnover number is attained.

Best Modes for Carrying Out the Invention

In the production process of the present invention, the raw material to be employed is an alkane or an alcohol. Preferred examples of the alkane include, but are not limited to, alicyclic alkanes such as adamantane and cyclohexane. Oxidation of such an alkane yields an alcohol, a diol, a polyol, or a ketone. In the case where the alkane is adamantane, the alcohol which can be obtained is 1-adamantanol or 2-adamantanol, the diol which can be obtained is 1,3-adamantanediol, the polyol which can be obtained is adamantanepolyol, or the ketone which can be obtained is 2-adamantanone. In the case where the alkane is cyclohexane, the alcohol which can be obtained is cyclohexanol, or the ketone which can be obtained is cyclohexanone.

Examples of the alcohol employed as a raw material in the present invention include, but are not limited to, 1-adamantanol, 2-adamantanol, and cyclohexanol. In the case where the alcohol is 1-adamantanol or 2-adamantanol, the diol which can be obtained is 1,3-adamantanediol, the polyol which can be obtained is adamantanepolyol, or the ketone which can be obtained is 2-adamantanone. In the case where the alcohol is cyclohexanol, the ketone which can be obtained is cyclohexanone.

In the present invention, a catalyst containing a transition metal element belonging to Groups 5 and 8 to 10 of the periodic table is employed as a sole catalyst. Examples of transition metal elements belonging to Groups 5 and 8 to 10 include vanadium, niobium, iron, cobalt, and nickel. In the present invention, a catalyst containing vanadium or cobalt is preferably employed. The vanadium to be employed preferably has a valence of 3 to 5, and the cobalt to be employed preferably has a valence of 2 or 3.

Examples of the vanadium compound which may be employed include acetylacetonatovanadyl [VO(acac)₂], ammonium metavanadate [NH₄VO₃], acetylacetonatovanadium [V(acac)₃], vanadyl sulfate [VOSO₄], vanadyl oxalate [VOC₂O₄], vanadium oxides (V₂O₅, V₆O₁₃, and VO₂), tris(isopropoxo)vanadyl [VO(OC₃H₇)₃], vanadium oxide stearate [CH₃(CH₂)₁₆COO]₂VO, vanadium oxychloride [VOCl₃], and vanadium oxide-TPP complex (TPP: 5,10,15,20-tetraphenyl-21H,23H-porphine). Of these, acetylacetonatovanadyl [VO(acac)₂], ammonium metavanadate [NH₄VO₃], acetylacetonatovanadium [V(acac)₃], and VO-TPP complex are preferred, with acetylacetonatovanadyl [VO(acac)₂] being particularly preferred, from the viewpoints of production of an oxygen-bearing compound at high yield, and attainment of high turnover number.

As used herein, the turnover number is obtained by use of the following equation: [the amount (mol) of adamantane consumed through reaction/the amount (mol) of an active metal (e.g., vanadium or cobalt) contained in the employed catalyst]. The turnover number is an index for reaction rate.

Examples of the cobalt compound which may be employed include cobalt (II) acetate [(CH₃COO)₂Co], acetylacetonatocobalt(II) [Co(acac)₂], acetylacetonatocobalt(III) [Co(acac)3], benzoylacetonatocobalt(II) [(C₆H₅COCH═C(O—)CH₃)₂Co], hexafluoroacetylacetonatocobalt [(CF₃COCH═C(O—)CF₃)₂Co], cobalt(II) chloride [COCl₂], cobalt(II) bromide [CoBr₂], cobalt(II) fluoride [CoF₂], cobalt(II) iodide [CoI₂], cobalt(II) nitrate [Co(NO₃)₂], phthalocyanine cobalt(II), cobalt(II) sulfate [COSO₄], cobalt oxides (CoO and Co₃O₄), cobalt(II) perchlorate [CoClO₄], cobalt(II) 2-ethylhexanoate [{CH₃(CH₂)₃CH(C₂H₅)COO}₂Co], cobalt(II) naphthenate, cobalt(II) thiocyanate [Co(SCN)₂], and cobalt-TPP complex (TPP: 5,10,15,20-tetraphenyl-21H,23H-porphine). Of these, acetylacetonatocobalt(II) [Co(acac)₂] is preferred, from the viewpoints of production of an oxygen-bearing compound at high yield, and attainment of high turnover number.

The present invention may employ a solid catalyst that a vanadium compound or cobalt compound is supported on a porous inorganic metallic carrier. Examples of the porous inorganic metallic carrier include silica, alumina, silica-alumina, titania, silica-titania, zeolite, titanosilicate, mesoporous silica, and mesoporous titania.

Properties of such a carrier vary in accordance with the type or production process thereof. The specific surface area is generally about 10 to 2,000 m²/g, preferably 50 to 1,500 m²/g, and the pore volume is generally about 0.01 to 2 cm³/g, preferably 0.1 to 1 cm³/g.

So long as the specific surface area and the pore volume fall within the above-described ranges, the catalytic activity is not reduced. The specific surface area and the pore volume can be obtained through, for example, the BET process on the basis of the volume of nitrogen gas adsorbed onto the catalyst [See J. Am. Chem. Soc., 60, 309 (1983)].

The production process of the present invention may employ, as an oxidizing agent, a gas containing molecular oxygen. Specific examples of the gas include air, oxygen, and a gas mixture obtained through dilution of air or oxygen with, for example, nitrogen, helium, or argon.

In the present invention, oxidation conditions in the case where an alkane is employed as a raw material are similar to those in the case where an alcohol is employed as a raw material. The reaction temperature is generally about 40 to 300° C., preferably 80 to 150° C. When the reaction temperature is 300° C. or lower, by-production of heavy components can be suppressed, resulting in an increase in selectivity, whereas when the reaction temperature is 40° C. or higher, the reaction rate increases, leading to an increas in production efficiency.

When the oxidizing agent is brought into a reactor, the oxidizing agent may be blown directly into the reaction mixture, or may be blown into a gas-phase portion of the reactor such that the oxidizing agent comes into contact with the surface of the reaction mixture. In the case that the oxidizing agent is to be blown directly into the reaction mixture, when the oxidizing agent is blown through an inlet provided beside a stirring blade, bubbles of the thus-blown agent are effectively involved in the vortex generated by the stirring blade, and dissolution of the oxidizing agent in the reaction mixture can be promoted. Oxidation may be performed at atmospheric pressure or reduced pressure, and, if necessary, oxidation may be performed under pressurized conditions. Such pressurization can promote dissolution of the oxidizing agent in the reaction mixture, thereby increasing the reaction rate.

In the present invention, oxidation may be of a batch type or a fixed-bed flow type. When batch-type oxidation is performed, the amount of the catalyst is generally about 0.00001 to 10 parts by mass, preferably 0.01 to 1 part by mass, on the basis of 100 parts by mass of a raw material (i.e., an alkane or an alcohol). When the catalyst amount is 0.00001 parts by mass or more, reaction is promoted, whereas when the catalyst amount is 10 parts by mass or less, treatment of the catalyst does not require a long period of time, and thus reaction efficiency is improved.

When batch-type oxidation is performed, the reaction time is generally about three minutes to 100 hours, preferably 1 to 20 hours. When the reaction time is three minutes or more, sufficient reaction conversion is attained, whereas when the reaction time is 100 hours or less, production efficiency is improved.

When fixed-bed-flow-type oxidation is performed, the reaction time represented by MHSV (mass hourly space velocity) is generally about 0.002 to 20 h⁻¹, preferably 0.05 to 1 h⁻¹. When the MHSV is 0.002 h⁻¹ or more, advantages are obtained in terms of production efficiency, whereas when the MHSV is 20 h⁻¹ or less, reaction proceeds sufficiently.

Whether oxidation is of a batch type or a fixed-bed flow type, if necessary, oxidation may be performed in a solvent. Examples of the solvent which may be employed include hydrocarbons such as dodecane and benzene; nitrites such as acetonitrile and benzonitrile; organic acids such as carboxylic acids (e.g., acetic acid, propionic acid, and butyric acid), halogenated carboxylic acids (e.g., chloroacetic acid), and sulfonic acids (e.g., methanesulfonic acid); esters such as methyl acetate, ethyl acetate, n-butyl acetate, and t-butyl acetate; and halogenated hydrocarbons such as dichloromethane and 1,1,2,2-tetrachloroethane. These solvents may be employed singly or in combination of two or more species. Of these solvents, carboxylic acids are preferred, with acetic acid and propionic acid being particularly preferred.

In order to enhance catalytic activity effectively, oxidation is carried out in the co-presence of one or more species selected from among a carboxylic acid, a sulfonic acid, and a Lewis acid. Examples of the sulfonic acid include methanesulfonic acid and toluenesulfonic acid. Examples of the Lewis acid include lanthanum triflate [La(OTf)₃] and europium triflate [Eu(OTf)₃]. Sulfuric acid or a solid acid (e.g., zeolite) may be employed in combination.

In the case where a carboxylic acid is employed as a solvent, when an additional acid is employed together with the carboxylic acid, the amount of the additional acid is generally regulated to about 0.001 to 10 mass %, preferably 0.01 to 1 mass % with respect to the carboxylic acid.

Depending on the type of the solvent, the following problem may arise; when the vapor of the solvent is intermingled with a molecular-oxygen-containing gas in the gas-phase portion in a reactor, there is a possibility of danger that the composition of the resultant gas mixture may fall within an explosive range. From the viewpoint of ensuring safety in industrial production, much preferably, reaction is performed such that the gas mixture composition falls outside the explosive range. Examples of processes for performing reaction in the present reaction system such that the gas mixture composition falls outside the explosive range include a process employing a noncombustible halogenated hydrocarbon solvent; a process in which the vapor pressure of a solvent is increased by increasing the reaction temperature so as to carry out reaction over the upper explosion limit; a process in which the vapor pressure of a solvent is reduced by decreasing the reaction temperature so as to carry out reaction below the lower explosion limit; a process in which the partial pressure of oxygen contained in a noncombustible gas mixture to be supplied is reduced such that the composition of the gas mixture falls outside an explosive range; and a process in which a gas having a high oxygen content is supplied in the liquid-phase portion of a reactor for highly efficient oxidation, and separately, an inert gas (e.g., nitrogen, carbon dioxide, or helium) is supplied to the gas-phase portion of the reactor such that the concentration of oxygen in the gas phase is reduced, and the composition of the resultant gas mixture falls outside an explosive range.

### EXAMPLES

The present invention will next be described in more detail by way of examples, which should not be construed as limiting the invention thereto.

### Example 1

Adamantane (10 mmol, 1.36 g) and acetylacetonatovanadyl [VO(acac)₂] (5 μmol, 1.3 mg) serving as a catalyst were dissolved in acetic acid (10 mL) placed in a three-neck flask, and oxygen (1 atm) was continuously blown into the flask at a flow rate of 10 mL/min under stirring with a stirrer, to thereby allow partial oxidation of adamantane (ADM) to proceed for six hours at 120° C. The resultant product was subjected to quantitative analysis by means of a gas chromatograph, and as a result, the product was found to contain 1-adamantanol (1-AdOH), 2-adamantanol (2-AdOH), 1,3-adamantanediol (1,3-(AdOH)₂), acetic acid esters of them, and 2-adamantanone (2-Ad=O). In the case of this product, adamantane conversion, total yield, and turnover number (TON) were found to be 37.0%, 25.8%, and 517, respectively. The results are shown in Table 1.

Table 1 also shows analysis results for the cases of the below-described Examples and Referential Examples. As used herein, the turnover number is obtained by use of the following equation: [amount (mol) of adamantane consumed through reaction/amount (mol) of active metal (e.g., vanadium or cobalt) contained in the employed catalyst]. The greater the turnover number, the higher the reaction rate.

### Example 2

The procedure of Example 1 was repeated, except that the amount of adamantane employed was changed to 5 mmol.

### Example 3

The procedure of Example 1 was repeated, except that the catalyst was replaced by acetylacetonatovanadium [V(acac)₃].

### Example 4

The procedure of Example 1 was repeated, except that the catalyst was replaced by ammonium metavanadate [NH₄VO₃].

### Example 5

The procedure of Example 1 was repeated, except that the catalyst was replaced by a vanadium oxide-TPP complex [VOTPP].

### Example 6

The procedure of Example 1 was repeated, except that the acetic acid serving as a solvent was replaced by propionic acid.

### Example 7

The procedure of Example 6 was repeated, except that the amount of the catalyst was changed to 10 μmol.

### Example 8

The procedure of Example 6 was repeated, except that the amount of the catalyst was changed to 1.3 μmol.

### Example 9

The procedure of Example 6 was repeated, except that the amount of adamantane employed was changed to 5 mmol, and the reaction temperature was changed to 100° C.

### Example 10

The procedure of Example 9 was repeated, except that methanesulfonic acid [CH₃SO₃H] was added in an amount of 0.004 mL.

### Example 11

The procedure of Example 9 was repeated, except that europium triflate [Eu(OTf)₃] was added in an amount of 10 μmol.

### Example 12

The procedure of Example 6 was repeated, except that the amount of the catalyst was changed to 10 μmol, and the adamantane was replaced by 1-adamantanol (5 mmol).

### Example 13

The procedure of Example 6 was repeated, except that the amount of adamantane employed was changed to 5 mmol, and the catalyst was replaced by Co(acac)₂.2H₂O.

### Referential Example 1

The procedure of Example 1 was repeated, except that the catalyst was replaced by a catalyst that vanadium (18 μmol) is supported on montmorillonite (i.e., a V/Mont. catalyst); the amount of adamantane employed was changed to 3 mmol; t-butyl acetate was employed as a solvent; and reaction was performed at 100° C. for 96 hours. The V/Mont. catalyst was formed through the catalyst preparation process described in Japanese Patent Application Laid-Open (kokai) No. 2004-2234. Specifically, the catalyst was obtained by adding an aqueous vanadium(III) chloride solution to montmorillonite (Kunipia F, product of Kunimine Industries Co., Ltd.), followed by ion exchange, filtration, washing with water, drying, and firing in air at 800° C.

### Referential Example 2

The procedure of Example 1 was repeated, except that the catalyst was replaced by NHPI (1 mmol) and VO(acac)₂ (50 μmol); the amount of acetic acid serving as a solvent was changed to 25 mL; and the reaction temperature was changed to 75° C.

[Table 1]

TABLE 1

| | | Raw | | Added | Reaction | Reaction | Yield (%) | | |
|---|---|---|---|---|---|---|---|---|---|
| | Catalyst | material | Solvent | acid | temp. | time | 1-AdOH | 2-AdOH | 2-AD = O |
| Ex. 1 | VO(acac)₂ | ADM | Acetic acid | | 120° C. | 6 h | 18.0 | 2.8 | 3.1 |
| | 5 μmol | 10 mmol | 10 mL | | | | | | |
| Ex. 2 | VO(acac)₂ | ADM | Acetic acid | | 120° C. | 6 h | 22.6 | 3.1 | 4.1 |
| | 5 μmol | 5 mmol | 10 mL | | | | | | |
| Ex. 3 | V(acac)₃ | ADM | Acetic acid | | 120° C. | 6 h | 22.3 | 3.0 | 3.8 |
| | 5 μmol | 5 mmol | 10 mL | | | | | | |
| Ex. 4 | NH₄VO₃ | ADM | Acetic acid | | 120° C. | 6 h | 10.1 | 1.5 | 1.5 |
| | 5 μmol | 5 mmol | 10 mL | | | | | | |
| Ex. 5 | VOTPP | ADM | Acetic acid | | 120° C. | 6 h | 15.3 | 2.7 | 2.4 |
| | 5 μmol | 5 mmol | 10 mL | | | | | | |
| Ex. 6 | VO(acac)₂ | ADM | Propionic acid | | 120° C. | 6 h | 33.1 | 4.3 | 7.0 |
| | 5 μmol | 10 mmol | 10 mL | | | | | | |
| Ex. 7 | VO(acac)₂ | ADM | Propionic acid | | 120° C. | 6 h | 31.6 | 3.9 | 7.5 |
| | 10 μmol | 10 mmol | 10 mL | | | | | | |
| Ex. 8 | VO(acac)₂ | ADM | Propionic acid | | 120° C. | 6 h | 36.2 | 4.6 | 5.5 |
| | 1.3 μmol | 10 mmol | 10 mL | | | | | | |
| Ex. 9 | VO(acac)₂ | ADM | Propionic acid | | 100° C. | 6 h | 15.5 | 2.3 | 2.0 |
| | 5 μmol | 5 mmol | 10 mL | | | | | | |
| Ex. 10 | VO(acac)₂ | ADM | Propionic acid | CH₃SO₃H | 100° C. | 6 h | 25.1 | 3.9 | 4.2 |
| | 5 μmol | 5 mmol | 10 mL | 0.004 mL | | | | | |
| Ex. 11 | VO(acac)₂ | ADM | Propionic acid | Eu(OTf)₃ | 100° C. | 6 h | 23.2 | 3.3 | 4.1 |
| | 5 μmol | 5 mmol | 10 mL | 10 μmol | | | | | |
| Ex. 12 | VO(acac)₂ | 1-AdOH | Propionic acid | | 120° C. | 6 h | 1.0 | 0.0 | 0.0 |
| | 5 μmol | 5 mmol | 10 mL | | | | | | |
| Ex. 13 | Co(acac)₂ | ADM | Propionic acid | | 110° C. | 6 h | 21.7 | 2.5 | 2.9 |
| | 5 μmol | 5 mmol | 10 mL | | | | | | |
| Ref. | V/Mont. | ADM | t-Butyl acetate | | 100° C. | 96 h | 38.0 | — | 14.0 |
| Ex. 1 | 18 μmol | 3 mmol | 10 mL | | | | | | |
| Ref. | NHPI | ADM | Acetic acid | | 75° C. | 6 h | 25.0 | — | — |
| Ex. 2 | 1 mmol, | 10 mmol | 25 mL | | | | | | |
| | VO(acac)₂ | | | | | | | | |
| | 50 μmol | | | | | | | | |

| | Yield (%) | | | Conversion | |
|---|---|---|---|---|---|
| | 1,3-Ad(OH)₂ | Others | Total | (%) | TON |
| Ex. 1 | 1.7 | 0.3 | 25.8 | 37.0 | 520 |
| Ex. 2 | 2.3 | 0.2 | 32.2 | 43.5 | 320 |
| Ex. 3 | 3.8 | 0.3 | 33.2 | 42.4 | 330 |
| Ex. 4 | 0.6 | 0.1 | 13.8 | 19.4 | 140 |
| Ex. 5 | 1.5 | 0.1 | 22.0 | 27.9 | 220 |
| Ex. 6 | 14.3 | 1.1 | 59.8 | 84.5 | 1200 |
| Ex. 7 | 21.1 | 0.7 | 64.8 | 88.1 | 650 |
| Ex. 8 | 7.9 | 0.1 | 54.2 | 70.1 | 4200 |
| Ex. 9 | 0.7 | 0.0 | 20.5 | 28.0 | 200 |
| Ex. 10 | 2.4 | 0.8 | 36.4 | 53.8 | 360 |
| Ex. 11 | 1.4 | 1.7 | 33.8 | 49.4 | 340 |
| Ex. 12 | 11.4 | 0.8 | 12.2 | 16.0 | 120 |
| Ex. 13 | 1.4 | 0.0 | 28.6 | 37.3 | 290 |
| Ref. | 40.0 | — | 92.0 | — | 150 |
| Ex. 1 | | | | | |
| Ref. | 34.0 | 12.0 | — | 98.0 | 10 |
| Ex. 2 | | | | | |

### INDUSTRIAL APPLICABILITY

The present invention provides a process for conveniently producing an alcohol, a diol, a polyol, or a ketone, which is useful as a solvent or an intermediate for producing various chemical products.

1. A process for producing an oxygen-bearing compound, comprising oxidizing an alkane in the presence of a catalyst containing at least one element selected from among transition metal elements belonging to Groups 5 and 8 to 10 of the periodic table, wherein the oxygen-bearing compound is an alcohol, a diol, a polyol, or a ketone.

2. A process for producing an oxygen-bearing compound, comprising oxidizing an alcohol in the presence of a catalyst containing at least one element selected from among transition metal elements belonging to Groups 5 and 8 to 10 of the periodic table, wherein the oxygen-bearing compound is a diol, a polyol, or a ketone.

3. A process for producing an oxygen-bearing compound as described in claim 1 or 2, wherein the transition metal element contained in the catalyst is vanadium.

4. A process for producing an oxygen-bearing compound as described in claim 3, wherein vandadium contained in the catalyst has a valence of 3 to 5.

5. A process for producing an oxygen-bearing compound as described in claim 4, wherein the catalyst is a vandadium compound having a vanadium valence of 3 to 5, or a solid catalyst formed of a porous inorganic metallic carrier bearing the vanadium compound.

6. A process for producing an oxygen-bearing compound as described in claim 1 or 2, wherein the transition metal element contained in the catalyst is cobalt.

7. A process for producing an oxygen-bearing compound as described in claim 6, wherein the catalyst is a cobalt compound having a cobalt valence of 2 or 3, or a solid catalyst formed of a porous inorganic metallic carrier bearing the cobalt compound.

8. A process for producing an oxygen-bearing compound as described in claim 5 or 7, wherein the porous inorganic metallic carrier is formed of silica, alumina, silica-alumina, titania, silica-titania, zeolite, titanosilicate, mesoporous silica, or mesoporous titania.

9. A process for producing an oxygen-bearing compound as described in claim 1, wherein oxidation is carried out by use of a molecular-oxygen-containing gas serving as an oxidizing agent.

10. A process for producing an oxygen-bearing compound as described in claim 1, wherein oxidation is carried out in the co-presence of one or more species selected from among a carboxylic acid, a sulfonic acid, and a Lewis acid.

11. A process for producing an oxygen-bearing compound as described in claim 1, wherein the catalyst containing a transition metal element is employed as a sole catalyst in an amount of 0.00001 to 10 parts by mass on the basis of 100 parts by mass of raw material.

12. A process for producing an oxygen-bearing compound as described in claim 1, wherein the alkane is adamantane, the alcohol is 1-adamantanol or 2-adamantanol, the diol is 1,3-adamantanediol, the polyol is adamantanepolyol, and the ketone is 2-adamantanone.
